(19) United States
(12) Patent Application Publication
(10) Pub. No.: US 2008/0269632 A1
(43) Pub. Date: Oct. 30, 2008

(54) Systems and Methods for Analyzing and Assessing Attention Deficit Hyperactivity Disorder

(75) Inventor: Steven M. Snyder, Boulder, CO (US)

Correspondence Address: SUTHERLAND ASBILL & BRENNAN LLP, 999 PEACHTREE STREET, N.E., ATLANTA, GA, 30309, US

(73) Assignee: LEXICOR MEDICAL TECHNOLOGY, LLC, Augusta, GA (US)

(21) Appl. No.: 11/877,357

(22) Filed: Oct. 23, 2007

Related U.S. Application Data
(60) Provisional application No. 60/853,569, filed on Oct. 23, 2006.

(51) Int. Cl. A61B 5/0476

(52) U.S. Cl. 600/544

(57) ABSTRACT

Embodiments of the invention can provide systems and methods for analyzing and assessing attention deficit hyperactivity disorder (ADHD) by integrating the use of electroencephalography (EEG), and ADHD diagnostic and assessment tools, such as an ADHD rating scale. Embodiments of the invention can provide some or all of the following improvements over conventional systems and methods, including: (1) Increased sensitivity, specificity, and overall accuracy; (2) Improved detection of ADHD; and (3) Distinguishing subjects with ADHD from subjects with a different disorder but having ADHD-like symptoms, such as attention and behavior symptoms similar to ADHD.

### RELATED APPLICATION

This application claims priority to U.S. Provisional application Ser. No. 60/853,569 entitled “Systems and Methods for Analyzing and Assessing Attention Deficit Hyperactivity Disorder (ADHD)”, filed on Oct. 23, 2006, the contents of which are incorporated by reference.

### TECHNICAL FIELD

The invention relates to detection of biological disorders. More particularly, the invention relates to systems and methods for analyzing and assessing attention deficit hyperactivity disorder (ADHD).

### BACKGROUND

The Centers for Disease Control and Prevention reports that the prevalence rates in the U.S. by state for attention deficit hyperactivity disorder (ADHD) diagnosis range from approximately 5-11% for children 4-17 years of age. In 2005, the U.S. Census Bureau reported that the population for children ages 5-17 years was about 53 million. Therefore, if each child ADHD patient in the estimated U.S. population averages at least one ADHD diagnostic scan, the potential annual market for ADHD analyses can be estimated in the range of approximately US$0.5 to US$1.2 billion.

However, recent studies have shown that of clinical subjects or patients having suspected ADHD-like symptoms, about 40% did not receive an ADHD diagnosis (Snyder et al., 2006; Quintana et al., 2006). In other words, clinical subjects or patients having ADHD-like symptoms represent a relatively larger population than those diagnosed with ADHD in a final clinical diagnosis.

Further, it has been reported that some ADHD symptoms can persist into adulthood for as many as 60-70% of children with ADHD (Bresnahan et al., 2005). Therefore, the potential market for ADHD analyses can extend to adults with either suspected ADHD-like symptoms or persistent ADHD symptoms.

Various conventional ADHD analysis, diagnostic, and assessment tools exist. The American Psychiatric Association (APA) publishes the Diagnostic and Statistical Manual of Mental Disorders (DSM, wherein the current version is referred to as the “DSM-IV”). The DSM and DSM-IV provide certain definitions and criteria for mental disorders which can support a clinician's diagnosis of a mental disorder in a subject or patient. The DSM-IV provides widely accepted criteria for a clinician to define ADHD in a subject or patient. As such, various analysis, diagnostic, and assessment tools have been developed based in part on professional guidelines used to assist clinicians in the implementation of DSM-IV criteria to analyze, diagnose, or otherwise assess ADHD in their subjects or patients.

An example of a diagnostic assessment tool is the ADHD behavior rating scale. The ADHD behavior rating scale is a recommended diagnostic assessment tool for diagnosing ADHD, wherein professional guidelines for its use by clinicians has been developed by, for example, the American Academy of Pediatrics (AAP, 2000). The ADHD behavior rating scale is designed to assist in the recognition of various attention and behavior symptoms of ADHD as defined by the DSM-IV. However, it is known that attention and behavior symptoms of ADHD are present with other disorders, such as oppositional defiant disorder, anxiety disorder, conduct disorder, mood disorder, adjustment disorder, reading disorder, and dyslexia (Cantwell, 1996; Goldman et al., 1998; Munoz-Milian and Casteel, 1989; Pary et al., 2002; Rucklidge and Tannock, 2002). The overlapping symptoms between ADHD and other disorders are reflected in the relative diagnostic accuracy of the ADHD rating scale when identifying ADHD within clinical samples of subjects or patients, the accuracy of which has been reported to range from approximately 60-79% (Snyder et al., 2006).

Another conventional ADHD diagnostic tool is based on a subject or patient's electroencephalogram (EEG) measurements. There are numerous studies which describe the association of EEG theta power and beta power changes with ADHD, such as Snyder and Hall (2006) and Barry et al. (2003). The theta/beta ratio, a function of both the EEG theta power and beta power, has been utilized by researchers studying ADHD in subjects and patients. The relative diagnostic accuracy for EEG detection of ADHD in a particular subject is typically reported at approximately 90% for ADHD versus normal controls. A “normal control” is a subject or patient who does not have ADHD, thus “normal controls” is a group of subjects or patients who do not have ADHD.

At least one patent relates to a method using EEG, specifically the theta and beta power measurements, for detection of ADHD in a subject versus normal controls. The method includes obtaining a total of four different recordings from the vertex location (CZ), determining the theta/beta ratio from each recording, and combining all of the theta/beta ratios to form an “Attentional Index.” One of the theta and beta power recordings is obtained with fixed gaze, while the other three recordings are obtained while the subject performs attention-requiring tasks, such as reading, listening, or copying geometric figures, appropriate for the subject's particular age group. The “Attentional Index” is compared to a previously-acquired normative database to provide a relative indication of the presence and severity of ADHD in the subject or patient. The relative diagnostic accuracy of this method is reported at approximately 88% for ADHD versus normal controls.

Even though conventional methods based on EEG measurements have relatively high diagnostic accuracy rates for ADHD, these methods are compared to normal controls, which may not be representative of actual clinical practice.

Therefore a need exists for systems and methods for analyzing and assessing attention deficit hyperactivity disorder (ADHD).

### SUMMARY OF THE INVENTION

Various embodiments of the invention can address some of all of the needs described above. Embodiments of the invention can provide systems and methods for analyzing and assessing attention deficit hyperactivity disorder (ADHD). In one embodiment, a system and method for analyzing and assessing ADHD can integrate the use of electroencephalography (EEG), and ADHD analysis, diagnostic, and assessment tools, such as an ADHD rating scale, to improve ADHD analysis and assessment. Embodiments of the invention can provide some or all of the following improvements over conventional systems and methods, including: (1) Increased sensitivity, specificity, and overall accuracy; (2) Improved detection of ADHD; and (3) Distinguishing subjects or patients with ADHD from subjects or patients with at least one different disorder but having ADHD-like symptoms, such as attention and/or behavior symptoms similar to ADHD. One embodiment of the invention can provide a method for assessing attention deficit hyperactivity disorder in a subject. The method can include receiving EEG data associated with a subject. The method can also include selecting a portion of the EEG data based at least in part on the number of artifacts in the EEG data. In addition, the method can include

determining at least one theta-beta ratio based at least in part on the selected EEG data. Moreover, the method can include standardizing the theta-beta ratio. Furthermore, the method can include receiving ADHD rating scale data associated with the subject. In addition, the method can include determining at least one ADHD rating scale score based at least in part on some or all of the ADHD rating scale data. Moreover, the method can include standardizing the at least one ADHD rating scale score. Furthermore, the method can include determining a probability the subject has ADHD based at least in part on both the standardized theta-beta ratio and standardized ADHD rating scale score.
Another embodiment of the invention can provide system for assessing attention deficit hyperactivity disorder in a subject. The system can include a data collection module operable to receive EEG data associated with a subject. The data collection module can be further operable to receive ADHD rating scale data associated with the subject. In addition, the system can include a processing module operable to determine at least one theta-beta ratio based at least in part on some or all of the EEG data. Furthermore, the processing module can be operable to determine at least one ADHD rating scale score based at least in part on the ADHD rating scale data. In addition, the processing module can be operable to standardize the at least one theta-beta ratio and the at least one ADHD rating scale score. The processing module can also be operable to determine a probability the subject has ADHD based at least in part on both the standardized theta-beta ratio and standardized ADHD rating scale score.
In yet another embodiment, a method for assessing attention deficit hyperactivity disorder in a subject can be provided. The method can include receiving EEG data and ADHD rating scale data associated with a subject. In addition, the method can include based at least in part on a selected portion of the EEG data, determining at least one theta-beta ratio. Furthermore, the method can include based at least in part on some or all of the ADHD rating scale data, determining at least one ADHD rating scale score. In addition, the method can include standardizing the theta-beta ratio and the at least one ADHD rating scale score. Moreover, the method can include based at least in part on both the standardized theta-beta ratio and standardized ADHD rating scale score, determining a probability the subject has ADHD.
In another embodiment of the invention, a method for assessing attention deficit hyperactivity disorder in a subject can be provided. The method can include receiving EEG data from about a CZ site associated with a subject. The method can also include receiving ADHD-IV rating scale data associated with the subject. Furthermore, the method can include based at least in part on a selected portion of the EEG data, determining at least one theta-beta ratio. In addition, the method can include based at least in part on some or all of the ADHD-IV rating scale data, determining at least one ADHD-IV rating scale score. Further, the method can include standardizing the theta-beta ratio and the at least one ADHD-IV rating scale score. The method can also include determining a probability the subject has ADHD, wherein the theta-beta ratio and the at least one ADHD-IV rating scale score are entered into a logistic regression model, and an output of the model comprises a probability the subject has ADHD.
Another embodiment of the invention can include a system for assessing attention deficit hyperactivity disorder in a subject. The system can include method a data collection module operable to receive EEG data from about a CZ site associated with a subject. The data collection module is further operable to receive ADHD-IV rating scale data associated with the subject. The system can also include a processing module operable to determine at least one theta-beta ratio based at least in part on a selected portion of the EEG data. The processing module is also operable to determine at least one ADHD-IV rating scale score based at least in part on some or all of the ADHD-IV rating scale data. In addition, the processing module is operable to standardize the theta-beta ratio and the at least one ADHD-IV rating scale score. Moreover, the processing module is operable to determine a probability the subject has ADHD, wherein the theta-beta ratio and the at least one ADHD-IV rating scale score are entered into a logistic regression model, and an output of the model comprises a probability the subject has ADHD.

One embodiment of the invention can provide an ADHD analysis and assessment tool which utilizes a statistical method, such as logistic regression, to integrate a set of EEG measurements for a particular subject with the subject's testing results from at least one ADHD rating scale, such as ADHD-IV (the ADHD Rating Scale-IV) or CRS-R (Conners' Rating Scales-Revised). The resultant analysis and assessment tool can be, for example, a comprehensive test which can yield a result or probability that a particular subject or patient is experiencing attention and/or behavior symptoms due to ADHD and not due to another disorder with ADHD-like symptoms.

One embodiment of the invention can provide an ADHD analysis and assessment tool which utilizes a non-linear-type analysis of EEG data rather than linear analysis, and statistically or mathematically combines the results of non-linear-type EEG analysis with other measures of ADHD testing, such as an ADHD rating scale. This embodiment can provide more reliable and relatively easier to obtain predictive information than the use of linear-type EEG data measures alone, such as those used in conventional systems and methods.

Another embodiment of the invention can provide an ADHD analysis and assessment tool which utilizes at least one predictive model in conjunction with at least one clinical database that includes data associated with patients or subjects with ADHD-like symptoms, but with only a portion of patients diagnosed with ADHD. Analysis and assessment results obtained using the predictive model can be cross-validated with the clinical database to determine the relative diagnostic accuracy of the predictive model.

In one example, cross-validation of a predictive model with at least one database in accordance with an embodiment of the invention can provide ADHD analysis and assessment improvements in sensitivity to approximately 87% and specificity to approximately 93% for subjects or patients with ADHD versus subjects or patients with other disorders having ADHD-like symptoms. In comparison, conventional ADHD diagnoses using rating scales alone may offer approximately 60-79% diagnostic accuracy. Conventional use of EEG measurements alone can offer similar diagnostic accuracy as rating scales alone, however, such techniques may not include integration into clinical applications with clinically representative samples. For instance, reported ADHD diagnostic accuracies for EEG data alone can be approximately 88% accurate using the “Attentional Index” described above, except this technique uses a relatively less challenging subject or patient sample of ADHD versus normal controls with no ADHD symptoms. In contrast, some embodiments of the invention can obtain relatively higher ADHD analysis and assessment accuracies for subjects or patients with ADHD versus subjects or patients with other disorders having ADHD-like symptoms, which can be a more representative scenario of actual clinical practice.

### BRIEF DESCRIPTION OF THE DRAWINGS

Embodiments of the invention can be better understood with reference to the following drawings.

FIG. 1 illustrates an example environment and system for analyzing and assessing ADHD in accordance with an embodiment of the invention.

FIG. 2 illustrates an example summary of improvements for an example system and method for analyzing and assessing ADHD in accordance with an embodiment of the invention.

FIG. 3 illustrates an example summary of improved rating scale results by integrating EEG for an example system and method for analyzing and assessing ADHD in accordance with an embodiment of the invention.

FIG. 4 is an example set of histograms of rating scale results without integration of EEG for a conventional or prior art system and method.

FIG. 5 is an example set of histograms of rating scale results integrated with EEG for an example system and method for analyzing and assessing ADHD in accordance with an embodiment of the invention.

FIG. 6 illustrates an example ROC curve for an example system and method for analyzing and assessing ADHD in accordance with an embodiment of the invention.

FIG. 7 illustrates an example set of ROC results by cutoff for the ROC curve shown in FIG. 6.

FIG. 8 is an example summary of cross-validation results for an example system and method for analyzing and assessing ADHD in accordance with an embodiment of the invention.

FIG. 9 illustrates an example summary of the prevalence of disorders in an example clinical database used with an example system and method for analyzing and assessing ADHD in accordance with an embodiment of the invention.

FIG. 10 illustrates an example comparative summary of results for an example system and method for analyzing and assessing ADHD in accordance with an embodiment of the invention.

FIG. 11 illustrates an example method for analyzing and assessing ADHD in accordance with an embodiment of the invention.

FIG. 12 illustrates another example method for analyzing and assessing ADHD in accordance with an embodiment of the invention.

### DETAILED DESCRIPTION OF EMBODIMENTS OF THE INVENTION

As used herein, the terms “subject” and “patient”, and their variants, can be used interchangeably without affecting the scope of embodiments of the invention. Furthermore, the terms “diagnostic” and “assessment”, and their variants, can be used interchangeably without affecting the scope of embodiments of the invention.

Various embodiments of the invention can provide systems and methods for analyzing and assessing attention deficit hyperactivity disorder (ADHD). In one embodiment, a system and method for analyzing and assessing ADHD can integrate the use of electroencephalography (EEG), and ADHD diagnostic and assessment tools, such as an ADHD rating scale. Embodiments of the invention can provide some or all of the following improvements over conventional systems and methods, including: (1) Increased sensitivity, specificity, and overall accuracy; (2) Improved detection of ADHD; and (3) Distinguishing subjects or patients with ADHD from subjects or patients with at least one different disorder but having ADHD-like symptoms, such as attention and/or behavior symptoms similar to ADHD.

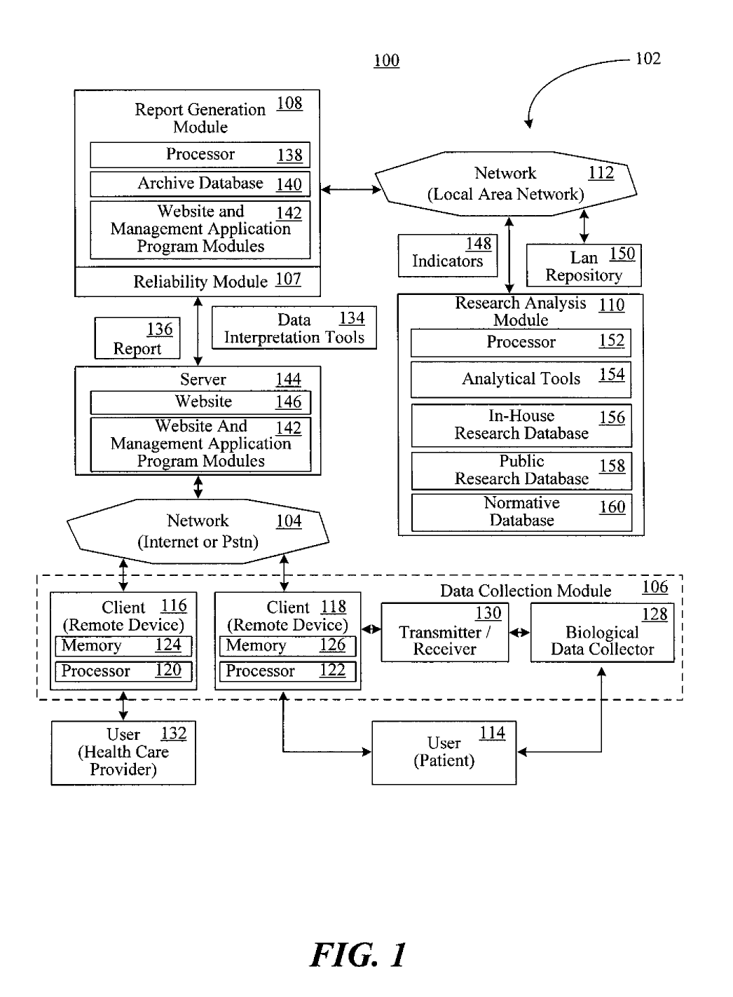

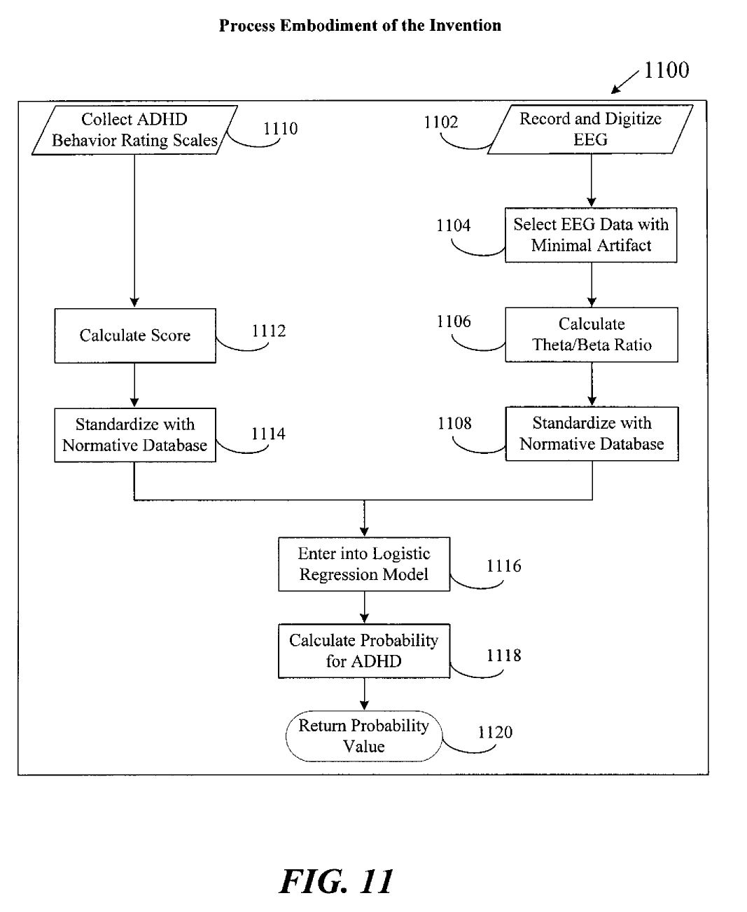

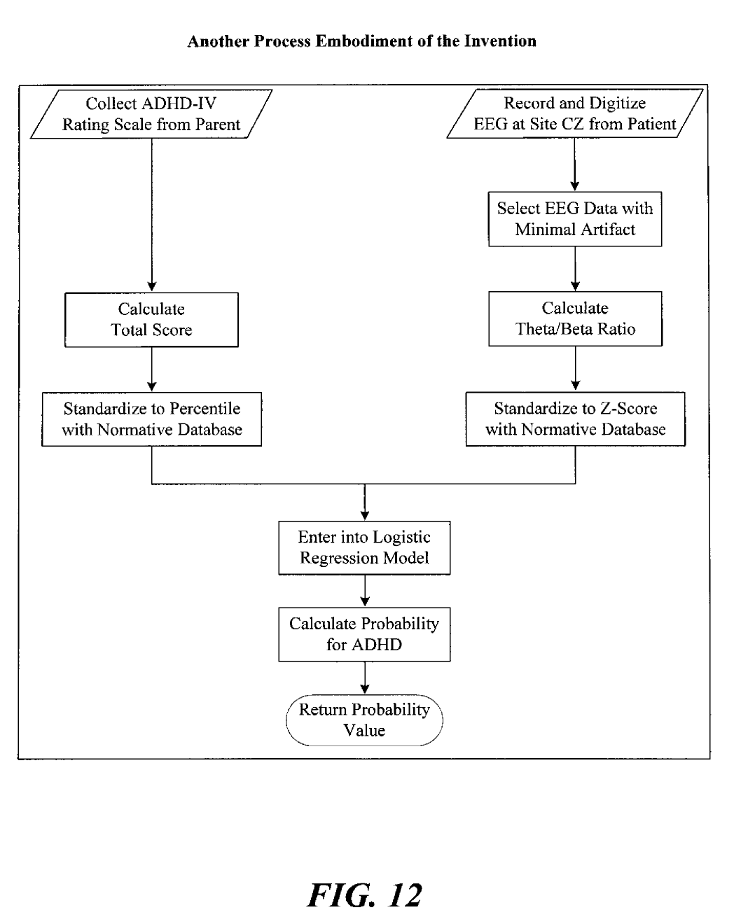

Systems for Analyzing and Assessing ADHD. FIG. 1 illustrates one example environment 100 for an example system 102 in accordance with an embodiment of the invention. The example environment and associated system components are similar to the environments and system components shown and described in commonly owned, co-pending applications U.S. Ser. No. 10/368,295, entitled “Systems and Methods for Managing Biological Data and Providing Data Interpretation Tools”, filed Feb. 18, 2003; and U.S. Ser. No. 11/053,627, entitled “Associated Systems and Methods for Managing Biological Data and Providing Data Interpretation Tools”, filed Feb. 8, 2005, the contents of which describe the associated system and related figure are hereby incorporated by reference. Using a system 102 illustrated in FIG. 1, various methods for analyzing and assessing ADHD in accordance with embodiments of the invention can be performed. In one example, the processes of FIGS. 11 and 12 can be implemented with the system 102 shown in FIG. 1. Other systems in accordance with other embodiments of the invention can include similar system components as shown in FIG. 1, or other components, elements, and modules.

By way of brief summary, the example environment 100 shown in FIG. 1 is a networked computer environment. The example system 102 can include a network 104, data collection module 106, report generation module 108, research analysis module 110, local network 112, and server 144. Other embodiments can include fewer or greater system elements or components, which may be in communication with each other as shown in FIG. 1 or may be in communication via other configurations in accordance with an embodiment of the invention. In any instance, the data collection module 106 can include at least one client-type device, such as 118, which can collect biological-type data, such as EEG data, from a user, such as 114. Rating scale-type data, such as from an ADHD rating scale, can be input or otherwise received or collected by a client-type device, such as 116. Data received or otherwise collected by client-type devices, such as 116 and 118, can be transmitted via a network, such as 104, to a server, such as 144, and/or a report generation module, such as 108. In either instance, the server 144 and/or report generation module 108 can include a website and management program module, such as 142, also known as a processing module. The website and management program module, such as 142, or otherwise known as a processing module may include a set of computer-executable instructions which can implement at least one predictive model operable to output at least one probability that a particular subject is suffering from ADHD versus other disorders with similar attention and/or behavior symptoms.

In one embodiment of the invention, a system, such as 102, can analyze and assess attention deficit hyperactivity disorder (ADHD) by integrating the use of electroencephalography (EEG), and ADHD diagnostic and assessment tools, such as an ADHD rating scale. Various output from the system, such as 102, can be used to facilitate analysis and assessment of ADHD in a subject or patient. Other analysis, diagnostic, and assessment tools, factors, and data can be implemented by a system, such as 102, in combination with some or all of the analysis, diagnostic, and assessment tools, factors, and data described above.

For example, in one embodiment of the invention, at least two factors can be input into a predictive model, such as a logistic regression model, to produce an output, such as a probability that a particular subject is suffering from ADHD versus other disorders with similar attention and/or behavior symptoms (“ADHD-like symptoms”). In this example, two factors can be at least one theta/beta ratio based at least in part on EEG data from a subject or patient, and at least one representative attention score for the subject or patient based at least in part on an ADHD behavior rating scale. The system, such as 102, can determine at least one theta/beta ratio from EEG data collected from a subject or patient, and can determine an attention score for the subject or patient based on an ADHD behavior rating scale. The system, such as 102, can combine the theta/beta ratio with the attention score for the particular subject or patient, and process the data in at least one predictive model, such as a logistic regression model, to generate an output or prediction of ADHD in the subject or patient.

In one embodiment, a system, such as 102, can implement at least one predictive model, such as a logistic regression model using stepwise selection, with a wide array of EEG variables, rating scales results, and other clinical data. The system, such as 102, can process the EEG variables, rating scales results, and other clinical data to determine an optimal predictive model. In other embodiments of the invention, particular EEG variables can be selected and combined with at least one representative attention score for a subject from an ADHD behavior rating scale. Selection of particular EEG variables in various embodiments can provide optimal predictive accuracy for a model, and therefore, selection of such variables for a predictive model may be preferred. EEG variables can include, but are not limited to, theta-beta ratio, absolute theta power, absolute beta power, relative theta power, and relative beta power.

In the example system 102 of FIG. 1, at least one predictive model can be implemented, for instance, by a website and management application module 142 at the server 144, or locally at a client-type level such as report generation module 108 with an associated website and management application module 142. Other modules, applications, or engines, local or server-level, may implement at least one predictive model with a system for analyzing and assessing ADHD in accordance with an embodiment of the invention.

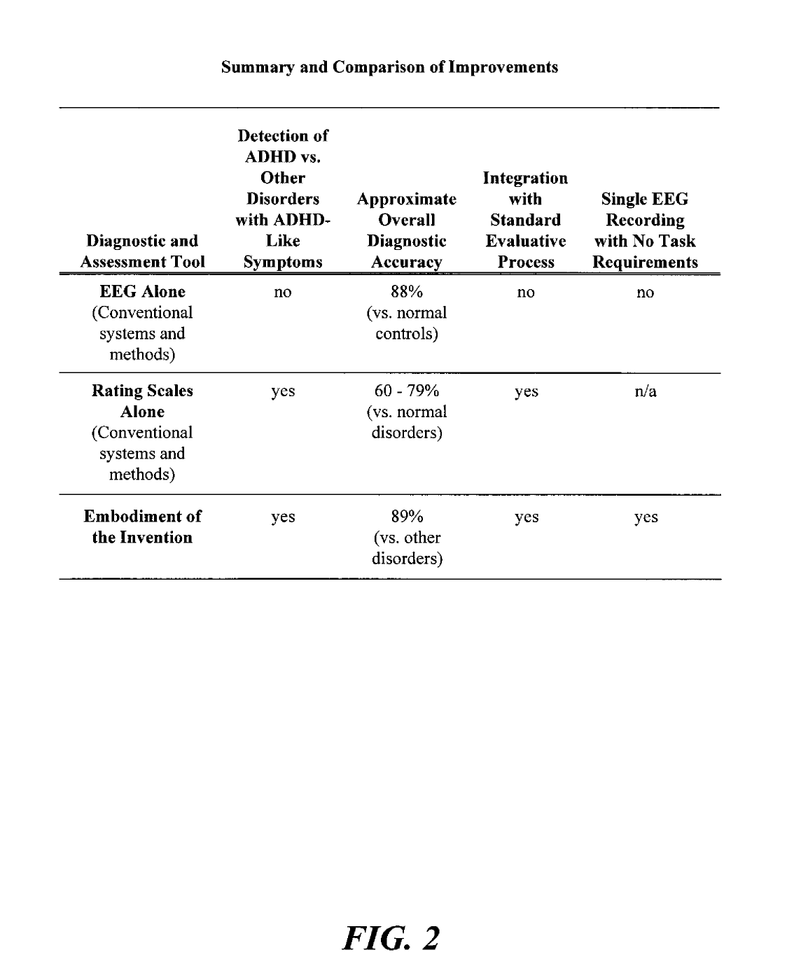

FIG. 2 summarizes improvements provided by an example system and method for analyzing and assessing ADHD in accordance with an embodiment of the invention over conventional systems and methods. As shown in FIG. 2, characteristics of conventional systems and methods are compared with characteristics of an example system and method for analyzing and assessing ADHD in accordance with an embodiment of the invention. In one embodiment, the combined use of EEG and rating scale data in analyzing and assessing ADHD can have improved assessment or diagnostic accuracy over conventional systems and methods, such as the use of EEG or conventional rating scales alone. In this example, an embodiment of the invention has an overall diagnostic accuracy of approximately 89% compared to the use of rating scales alone, which has an overall diagnostic accuracy of approximately 60-79%. The overall diagnostic accuracy of EEG alone is approximately 88%, however, this accuracy is only versus normal controls, and is not representative of actual clinical practice.

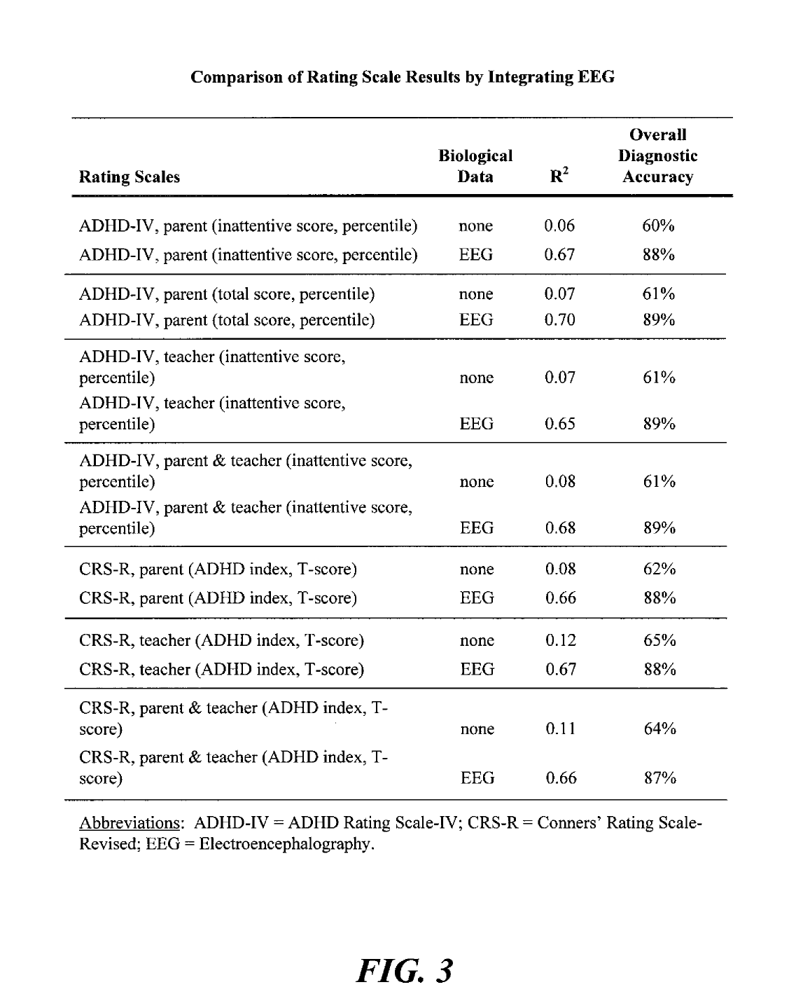

FIG. 3 illustrates various statistical improvements of an example system and method for analyzing and assessing ADHD in accordance with an embodiment of the invention. In the embodiment shown in FIG. 3, the example system and method integrates EEG data and rating scales results. The statistical improvements of the example system and method are shown compared to statistics for the use of a particular rating scale alone. In this embodiment, a logistic regression model was applied by the example system and method to detect ADHD in a clinical sample of patients with a variety of disorders, wherein each patient exhibited ADHD-like symptoms. Furthermore, FIG. 3 represents statistical data from seven different example integrations of EEG data with different rating scales, with each example showing a statistical improvement over use of the rating scale alone. By way of example, the embodiment of the system and method of FIG. 3 includes EEG data integrated with different rating scales, such as ADHD-IV and CRS-R. In other embodiments of the invention, any ADHD rating scale can be integrated with EEG data to provide an ADHD analysis and assessment tool for a patient or subject. Rating scale examples include the ADHD index of CRS-R, inattentive score of ADHD-IV, and total score of ADHD-IV. Informants for the rating scales included parents, teachers, and a combination of the two groups.

The relatively higher R² values, from a minimum of 0 to a maximum of 1, in FIG. 3 can indicate that more of the variation can be explained by the example predictive model. Furthermore, the relatively higher overall diagnostic accuracies shown in FIG. 3 can indicate that the sensitivity and specificity will, on average, be higher than conventional systems and methods. It can be seen in FIG. 3 that the integration of EEG data together with each rating scale can offer an improvement in R² value and overall diagnostic accuracy. The average improvement in R² value, after integration of EEG data with a rating scale, is approximately 0.59. The average improvement in overall diagnostic accuracy, after integration of EEG data with a rating scale, is approximately 26%.

For further illustration of various statistical support for embodiments of the invention, one particular rating scale, ADHD-IV, has been selected to further illustrate improvements over conventional systems and methods.

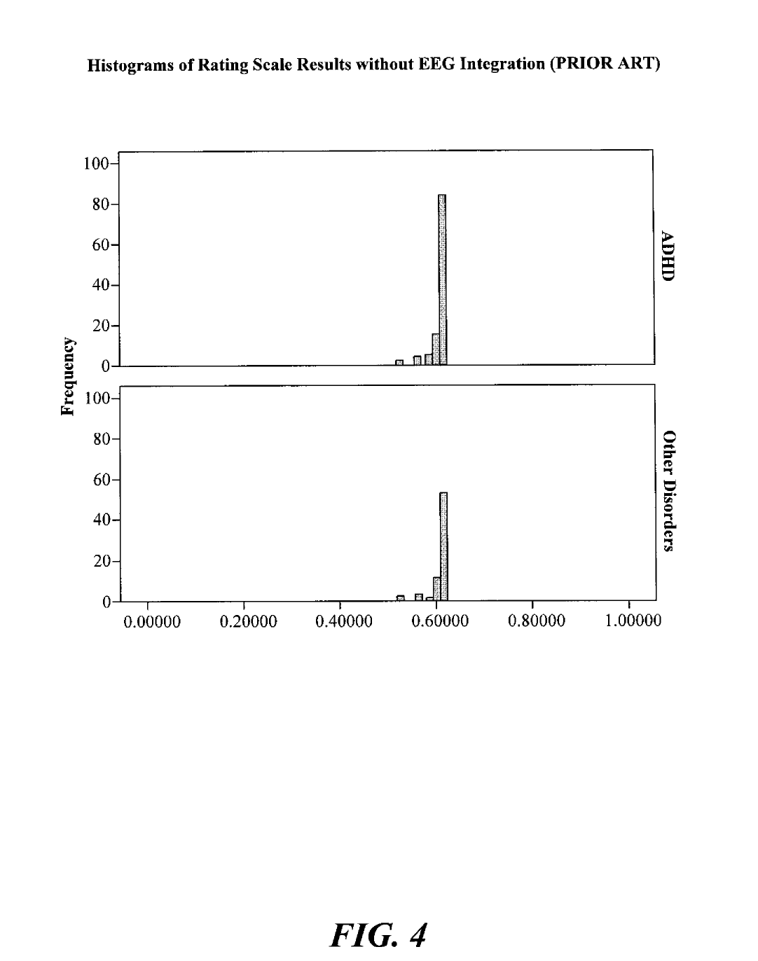

FIG. 4 illustrates a set of histograms for the application of a conventional method that uses an ADHD rating scale alone (ADHD-IV, parent, total score, percentile) to analyze and assess ADHD. The separation of ADHD patients from non-ADHD patients aged 6-21 in a clinical database (N=185) is shown in FIG. 4.

As can be observed in the histograms of FIG. 4, application of the rating scale alone (without EEG data integration) to analyze and assess patients does not separate ADHD patients from patients with different disorders and having ADHD-like symptoms. This outcome is reflected in the data shown in the histograms of FIG. 4, which have a R² value of approximately 0.07, and an overall diagnostic accuracy of approximately 61%.

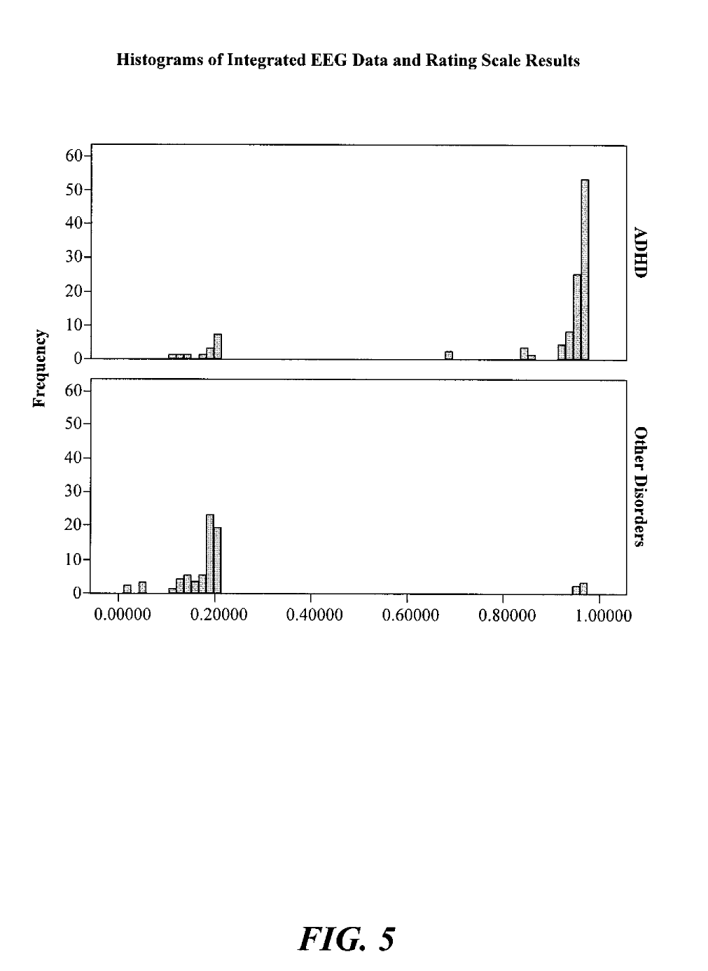

FIG. 5 illustrates a set of histograms for the application of integrated EEG data with an ADHD rating scale (ADHD-IV, parent, total score, percentile) in an example system and method for analyzing and assessing ADHD in accordance with an embodiment of the invention. The relative separation of ADHD and non-ADHD patients with an embodiment of the invention is shown in FIG. 5.

As can be observed in FIG. 5, an embodiment of the invention can distinguish a majority of subjects as patients having ADHD from the majority of patients who do not have ADHD. This separation is reflected in the data shown in the histograms of FIG. 5, which have a R² value of approximately 0.70, and an overall diagnostic accuracy of approximately 89%.

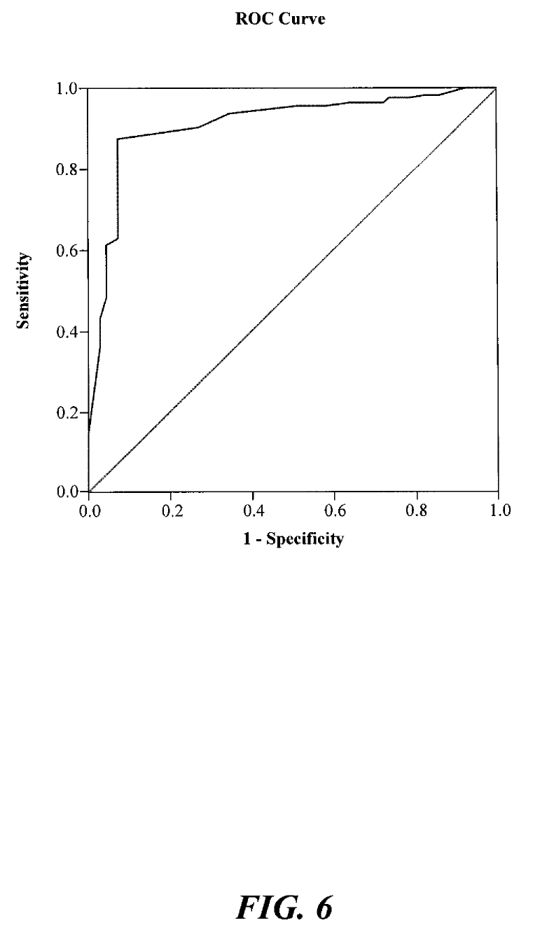

In one embodiment of the invention, an example system and method for analyzing and assessing ADHD can implement a predictive model to determine a probability between 0 and 1 for a particular subject or patient, wherein the probability represents or is otherwise indicative of the subject or patient's membership in the ADHD population. The evaluated clinical sample for FIG. 5 comprises clinical patients who had presented suspected attention and/or behavior problems (ADHD-like symptoms). Of the clinical patients in this clinical sample, approximately 61% were diagnosed with ADHD, and approximately 39% were diagnosed with other disorders but not ADHD (see below for more details on the clinical database). In this embodiment, a receiver operating characteristic (ROC) curve can be generated by an example system, such as 102 in FIG. 1, for analyzing and assessing ADHD. A suitably qualified clinician can use an ROC curve and tabulated results to determine a probability which represents or is otherwise indicative of a particular subject or patient's membership in the ADHD population. An ROC curve and tabulated results can provide sensitivity and specificity values for each probability cutoff chosen. An example ROC curve for the above example is shown in FIG. 6. In this embodiment, the ROC curve (upper curve) obtained from data from a clinical database is shown in FIG. 6 compared to the standard reference line for an ROC curve (diagonal line).

Generally, the further the ROC curve (upper curve) lies above the reference line (diagonal line), the more accurate the diagnostic and assessment data. In this example, data and results from a clinical database are shown as the ROC curve (upper curve). The quantitative area under the ROC curve (upper curve) is approximately 0.91, which means that in the clinical sample of patients with attention and behavior problems, there is approximately a 91% probability that the diagnostic and assessment data result for a randomly chosen ADHD patient will exceed the result for a patient without ADHD.

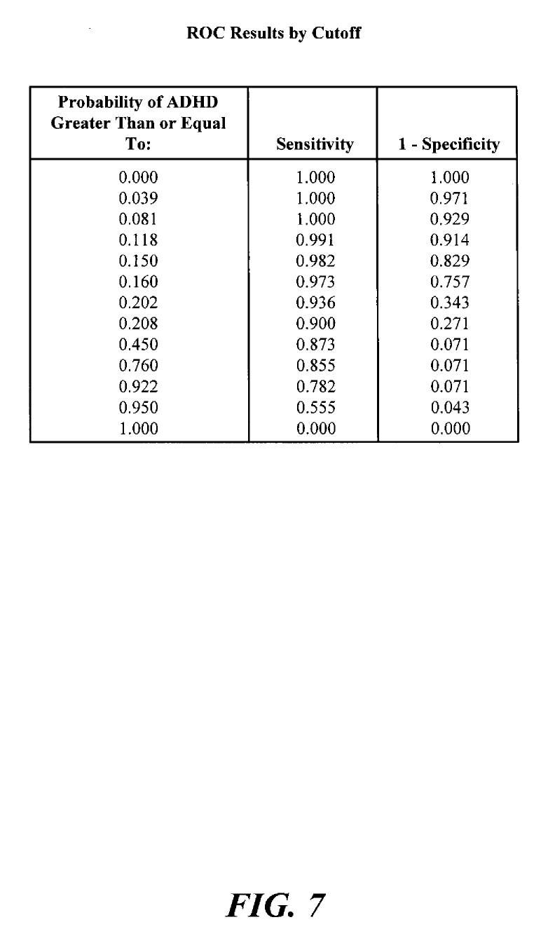

An example of the tabulated results by cutoff for the ROC curve shown in FIG. 6 is illustrated in FIG. 7.

In one embodiment, each data source such as EEG data and rating scale data can be verified by the system, such as 102 in FIG. 1, for inclusion in a predictive model in accordance with an embodiment of the invention. In some instances, if data is not verified, a predictive model can be “overfit,” which may limit the generalizability of the predictive model. Overfitting of a predictive model can occur if enough predictor variables are included in the predictive model, such that the observed overall diagnostic accuracy approaches a relatively high level for that sample due to recognition of random factors specific to that sample. Any number of analytical techniques can be implemented by the system, such as 102, to address overfitting, for example, cross-validation. The statistics shown in FIG. 7 illustrate that the example predictive model in this embodiment is not overfit, and there is suitable statistical support in the prediction of an ADHD diagnosis using the predictive model.

Average Improvement in R² Value. The results in FIG. 7 illustrate that the integration of EEG data with different rating scales can offer a relative improvement in overall diagnostic accuracy and R² value over the use of rating scales alone. That is, there is an improvement in the predictive accuracy of ADHD by the integration of EEG data with rating scale results. In the embodiment shown in FIG. 7, the average improvement in R² value with the addition of EEG is approximately 0.59. The average improvement in overall accuracy with the addition of EEG is approximately 26%.

Forward Stepwise Logistic Regression. In one embodiment, forward stepwise logistic regression method can be used in an example system and method for analyzing and assessing ADHD to verify the inclusion of some or all variables in an associated predictive model. In this example, the system and method can statistically select and test each variable in succession, and only variables making a significant contribution would ultimately be selected for inclusion within the predictive model. By only including significant variables, this system and method can reduce the possibility of overfitting. Both EEG data and rating scales results contributed significant information to the model, indicated by significant changes in 2-log-likelihood (P<0.05). Therefore, based at least on this statistic, the inclusion of all variables in this example predictive model is statistically valid.

Backward Stepwise Logistic Regression. In one embodiment, the example predictive model can be verified with backward stepwise logistic regression. In this example, a backward stepwise logistic regression method statistically selects and tests each variable in succession, and only variables making a significant contribution would ultimately be selected for inclusion within the predictive model. This is similar to the forward stepwise logistic regression method described above. Since the backward stepwise logistic regression method selected the same significant variables as the example forward stepwise logistic regression method, this indicates a good model of the data. In other words, application of a different logistic regression method (backward) resulted in the same selection of EEG data and rating scales results as the forward stepwise logistic regression method, which provides confirmation of the example predictive model and verification that there was no overfitting.

Goodness of Fit. In one embodiment, a goodness of fit method can determine whether an example predictive model uses data to sufficiently describe a prediction. In this example, the goodness of fit of an example predictive model was checked with the Hosmer-Lemeshow statistic, which provided a P-value of approximately 0.55. A P-value greater than approximately 0.5 predicts goodness of fit. Therefore, the goodness to fit model verified that the example predictive model adequately fit the data.

R² Statistic. In one embodiment, the R² statistic can predict whether variation of the outcome (ADHD diagnosis) is covered by the example predictive model. In this example, the Nagelkerke R² statistic was determined to be approximately 0.70, which indicates that approximately 70% of the variation in the DSM-IV diagnosis of ADHD patients versus patients with other disorders can be explained by the model predictors.

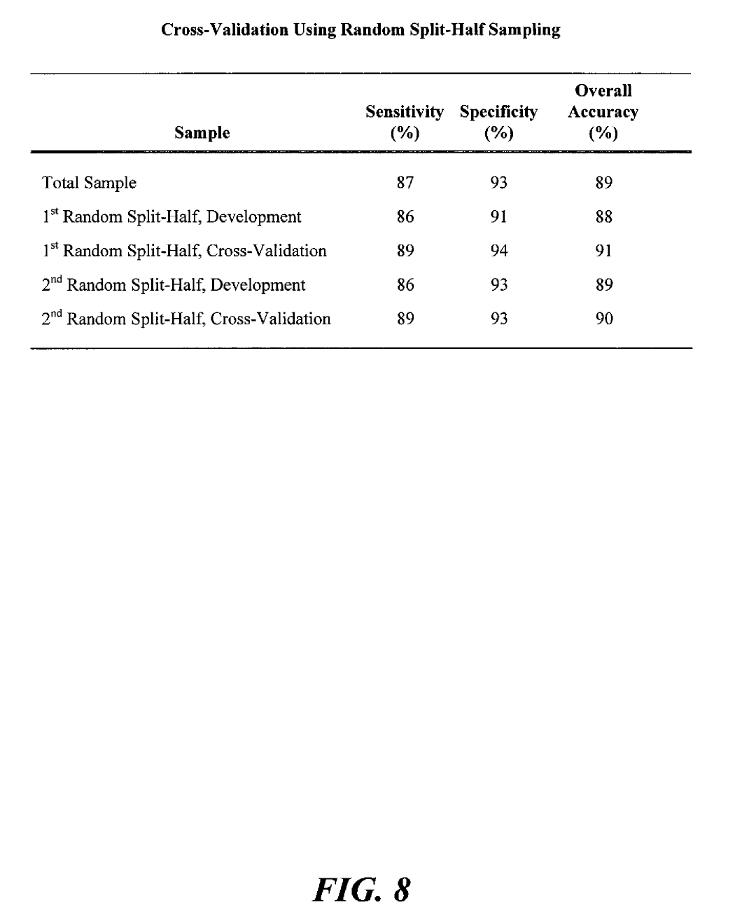

Cross-Validation. With logistic regression modeling, the standard cutoff can be at a calculated probability of approximately 0.5. In this embodiment, a clinical database (n=185) was subjected to random split-half sampling to develop and then cross-validate the example predictive model with separate samples (with 0.5 cutoff as reference). This technique was repeated for further verification. The results of this method are shown in FIG. 8.

The consistency of the results shown in FIG. 8 provides support that the integration of EEG data and ratings scale results is appropriate and the example predictive model is well fit.

Clinical Database. The example predictive model described above can be compared with demographic data in at least one clinical database. In one embodiment, demographic data supporting a population sample can be compared to demographic data in at least one clinical database to determine whether the particular population sample is representative of clinical practice. In one example of a clinical database, demographic data representing patients at four clinics, such as two university child psychiatric sites, one private pediatric site, and one private child psychiatric site, can be collected for a period of time, for instance, Apr. 8, 2004 to Jul. 30, 2005. Particular subjects can be included in the demographic data if a parent or school official suspected a child/adolescent might have ADHD. A clinical standard used for classification of patients was DSM-IV diagnosis by a pediatrician or psychiatrist with support from a semi-structured clinical interview.

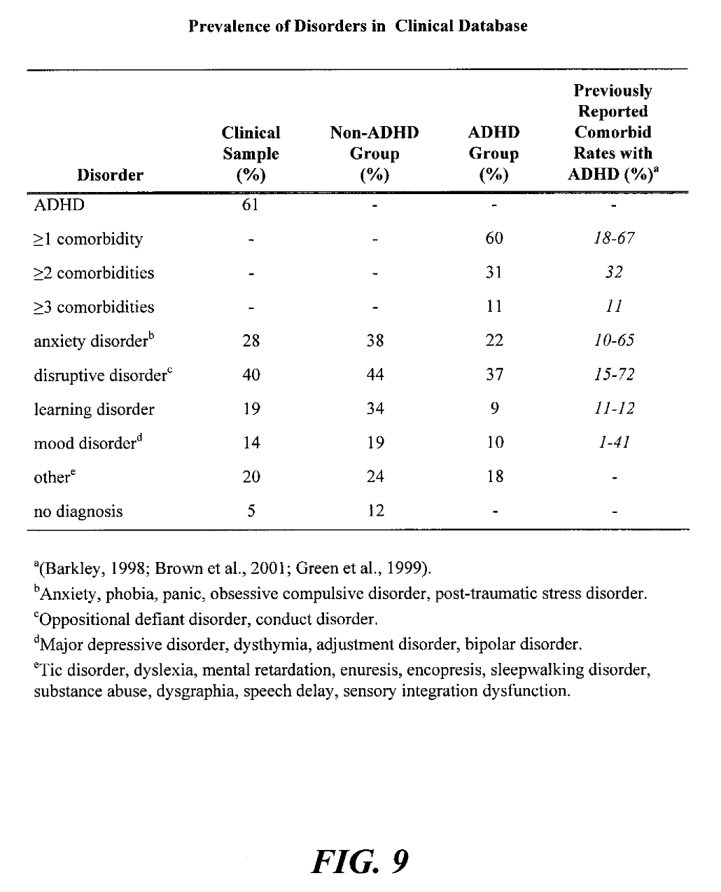

As shown in FIG. 9, the comparison of demographic data for a population sample is consistent with demographic data in an example ADHD clinical database. In particular, the demographic data is consistent for comorbid rates for anxiety, disruptive, mood, and learning disorders as compared with the comorbid rates observed in clinical samples covered in expert reviews (Barkley 1998; Brown et al., 2001; Green et al., 1999). In another embodiment, a population sample can be compared with demographic data in a clinical database, wherein the demographic data includes reported rates of one to three comorbidities. Additionally, learning disorder prevalence with specific disorders (conduct disorder, oppositional defiant disorder, ADHD, major depressive disorder, or dysthymic disorder) is approximately 15%, consistent with the DSM-IV report of 10-25% (APA, 1994).

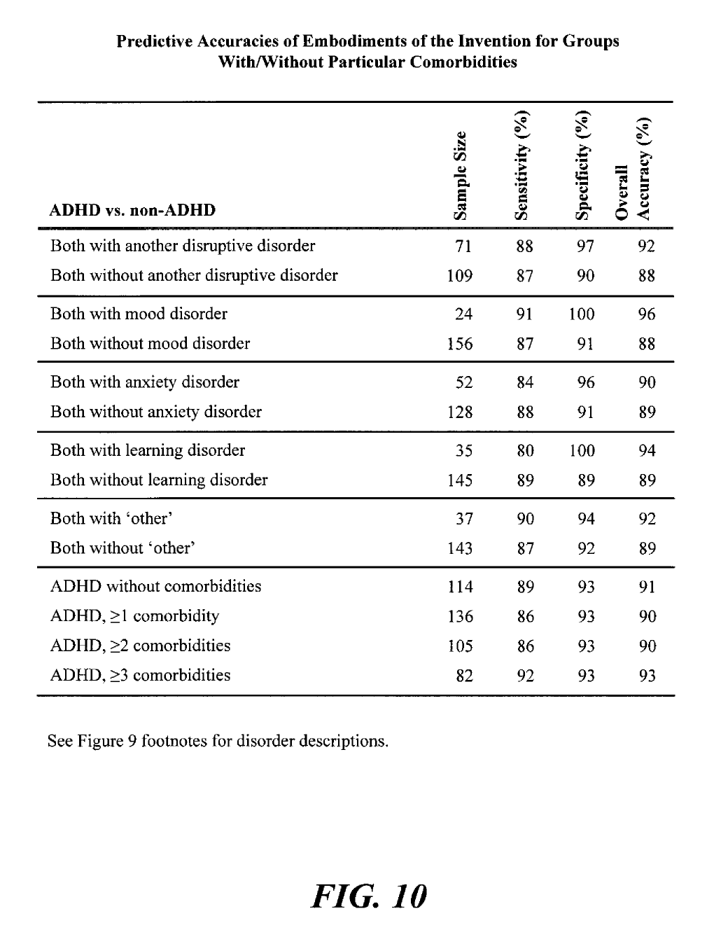

Predictive Accuracies in Presence or Absence of Particular Comorbidities. FIG. 10 illustrates predictive accuracies of an example system and method in accordance with an embodiment of the invention when applied to two different groups, one group having a particular comorbid condition, and the other group without the particular comorbid condition.

The results shown in FIG. 10 illustrate the relative accuracies of a system and method in accordance with an embodiment of the invention, which are consistent for ADHD in the presence or absence of particular comorbidities. For instance, ‘ADHD with an anxiety disorder’ versus ‘non-ADHD with an anxiety disorder’ were classified with a sensitivity of 84%, specificity of 96%, and overall accuracy of 90%, which are consistent with results of ADHD vs. non-ADHD in the absence of an anxiety disorder: sensitivity of 88%, specificity of 91%, and overall accuracy of 89%. Therefore, this example system and method in accordance with an embodiment of the invention has relatively high predictive accuracy when applied with a clinical database representative of clinical practices that provide care for patients with attention and behavior symptoms.

Some or all of the techniques and methodologies described with respect to FIGS. 5-10 can be implemented by a system, such as 102 in FIG. 1, to verify, modify, or otherwise change a predictive model to analyze and assess ADHD in accordance with an embodiment of the invention.

Methods for Analyzing and Assessing ADHD. Example embodiments of methods for analyzing and assessing ADHD are described in FIGS. 11 and 12. The elements of the methods shown in FIGS. 11 and 12 are shown by way of example, and other methods in accordance with an embodiment of the invention can include fewer or greater elements, and may perform elements in another sequential order than shown or described herein. As described above, an example system to implement these methods and other embodiments is shown as 102 in FIG. 1 and described above.

FIG. 11 illustrates a method 1100 for collecting and analyzing EEG data, and for collecting ADHD behavior rating scale data. The method 1100 in FIG. 1100 begins at block 1102. Blocks 1102-1108 represent a method to collect and analyze EEG data.

In block 1102, EEG data from a patient is recorded and digitized. In this embodiment, at least one electrode capable of collecting EEG data is mounted to a subject or patient's body. For example, an electrode can be placed at site CZ of a patient's body, located using the International 10-20 system of electrode placement. In this example, the patient's body can be cleaned using an appropriate EEG preparation cleaner and alcohol. Once the electrode is suitably placed, a syringe can be used to apply conductive gel to the patient's scalp in the selected site. The electrode site can be checked to ensure that a relatively accurate reading can be obtained from that site.

In one embodiment, EEG data can be collected with the patient or subject's eyes opened (fixed gaze). Typically, 10 minutes of EEG data (315 epochs) are collected.

Block 1102 is followed by block 1104, in which EEG data is selected with minimal artifacts. In this embodiment, the collected EEG data is screened for artifacts, and any affected epochs are removed from the EEG data set.

Block 1104 is followed by block 1106, in which based at least in part on the EEG data, a theta/beta ratio is determined. In this embodiment, analysis of the collected EEG data set is performed to calculate a theta/beta ratio. For example, a theta/beta ratio can be calculated by first computing the percent power of the Theta and Beta1 Bands. Bands are derived using the “D-Base Bands” Delta1 (0-2 Hz), Delta2 (2-4 Hz), Theta (4-8 Hz), Alpha (8-13 Hz), Beta1 (13-21 Hz), and Beta2 (21-32 Hz). The theta/beta ratio can be calculated by using the formula:

T/B₁

Wherein:
T=Percent Power Theta at CZ
B₁=Percent Power Beta1 at CZ

Block 1106 is followed by block 1108, in which the theta/beta ratio is standardized. In this embodiment, the theta/beta ratio can be standardized to a Z-score using a normative database by age. In this example, a final variable is set to categorical using a 1.5 Z-score cutoff.

The method 1100 continues at block 1110. Blocks 1110-1114 represent a method to collect ADHD behavior rating scale data. In block 1110, rating scale data associated with the subject or patient is received. In this embodiment, an ADHD behavior rating scale associated with the subject or patient can be completed by an informant, such as a parent or a teacher.

Block 1110 is followed by block 1112, in which based at least in part on the rating scale data, a score is determined.

Block 1112 is followed by block 1114, in which the score is standardized. In this embodiment, the score can be standardized to a T-score or percentile using a normative database by age and gender.

In one embodiment, various ADHD rating scales can provide sufficient information on attention and behavior symptoms, and the data from such ADHD rating scales can have a significant effect on an applied logistic regression model. Examples of these rating scales can include, but are not limited to, an inattentive score or the total score of the ADHD-IV, or the ADHD index of the CRS-R, with each scale rating completed by teacher or parent. Results from other ADHD rating scales can be used with other embodiments of the invention. In at least one embodiment, the total score of the parent version of the ADHD-IV can be utilized. In other embodiments, rating scales can be collected from multiple informants, such as from both a teacher and a parent. The resultant rating scale scores can be converted to percentiles or T-scores, and the results from different informants can be averaged to produce a final score. The final score can be entered into a predictive model in accordance with an embodiment of the invention.

The method 1100 continues at block 1116, in which at least one output from blocks 108 and 1114 is received, and input into a predictive model. In this embodiment, once data collection has been performed in blocks 1102-1108 (collecting one EEG recording of 10 minutes of eyes open, fixed gaze data at CZ) and blocks 1110-1114 (collection of ADHD rating scales), the data are analyzed and input into a predictive model, such as a logistic regression model.

Block 1116 is followed by block 1118, in which an output or probability is determined. In this embodiment, the result of the example logistic regression model can be output as a probability that the subject or patient in question is suffering from ADHD. The probability result can be interpreted by a clinician using an ROC curve and table representing a clinical database of ADHD patients and patients with other disorders but with similar attention and behavior symptoms (ADHD-like symptoms). The ROC curve and table can provide sensitivity and specificity results for the predictive model, which can be interpreted by the clinician when integrating the result from the predictive model together with the clinician's complete clinical evaluation and assessment tests.

Block 1118 is followed by block 1120, in which an output or probability is provided. The method 1100 ends at block 1120.

Another process embodiment of the invention, similar to that shown and described in FIG. 11, is shown in FIG. 12.

While the above description contains many specifics, these specifics should not be construed as limitations on the scope of the invention, but merely as exemplifications of the disclosed embodiments. Those skilled in the art will envision many other possible variations that are within the scope of the invention.

The claimed invention is:

1. A method for assessing attention deficit hyperactivity disorder in a subject, the method comprising:
receiving EEG data associated with a subject;
selecting a portion of the EEG data based at least in part on the number of artifacts in the EEG data;
based at least in part on the selected EEG data, determining at least one theta-beta ratio; and
standardizing the theta-beta ratio;
receiving ADHD rating scale data associated with the subject;
based at least in part on some or all of the ADHD rating scale data, determining at least one ADHD rating scale score;
standardizing the at least one ADHD rating scale score; and
based at least in part on both the standardized theta-beta ratio and standardized ADHD rating scale score, determining a probability the subject has ADHD.

2. The method of claim 1, wherein at least a portion of the EEG data is collected from the subject's body from about the CZ site location using the International 10-20 system of electrode placement.

3. The method of claim 1, wherein at least a portion of the EEG data is collected while the subject's eyes are opened in a fixed gaze.

4. The method of claim 1, wherein the number of artifacts is a predefined threshold.

5. The method of claim 1, wherein the theta-beta ratio is determined by dividing the percent power of a theta band associated with the selected EEG data by the percent power of a beta1 band associated with the selected EEG data, wherein the theta band is about 4 Hz to about 8 Hz, and the beta1 band is about 13 Hz to about 21 Hz.

6. The method of claim 1, wherein the theta-beta ratio is standardized to a Z-score using a normative or clinical database by at least age.

7. The method of claim 1, wherein the ADHD rating scale data can comprise data from at least one of the following: an inattentive score or total score of a ADHD-IV rating scale, or an ADHD index of a CRS-R rating scale.

8. The method of claim 1, wherein the at least one ADHD rating scale score is standardized to a normative or clinical database by at least age and gender.

9. The method of claim 1, wherein the probability determination is facilitated with a logistic regression model.

10. The method of claim 1, wherein a receiver operating characteristic (ROC) curve can be used to output the probability determination.

11. A system for assessing attention deficit hyperactivity disorder in a subject, the system comprising:
a data collection module operable to:
receive EEG data associated with a subject;
receive ADHD rating scale data associated with the subject;
a processing module operable to:
determine at least one theta-beta ratio based at least in part on some or all of the EEG data;
determine at least one ADHD rating scale score based at least in part on the ADHD rating scale data;
standardize the at least one theta-beta ratio and the at least one ADHD rating scale score; and
determine a probability the subject has ADHD based at least in part on both the standardized theta-beta ratio and standardized ADHD rating scale score.

12. The system of claim 11, wherein the EEG data is collected from the subject's body from about the CZ site location using the International 10-20 system of electrode placement.

13. The system of claim 11, wherein the EEG data is collected while the subject's eyes are opened in a fixed gaze.

14. The system of claim 11, wherein the number of artifacts is a predefined threshold.

15. The system of claim 11, wherein the theta-beta ratio is determined by dividing the percent power of a theta band associated with the selected EEG data by the percent power of a beta1 band associated with the selected EEG data, wherein the theta band is about 4 Hz to about 8 Hz, and the beta1 band is about 13 Hz to about 21 Hz.

16. The system of claim 11, wherein the theta-beta ratio is standardized to a Z-score using a normative or clinical database by at least age.

17. The system of claim 11, wherein the ADHD rating scale data can comprise data from at least one of the following: an inattentive score or total score of a ADHD-IV rating scale, or an ADHD index of a CRS-R rating scale.

18. The system of claim 11, wherein the at least one ADHD rating scale score is standardized to a normative database by at least age and gender.

19. The system of claim 11, wherein the probability determination is facilitated with a logistic regression model.

20. The system of claim 11, wherein a receiver operating characteristic (ROC) curve can be used to output the probability determination.

21. A method for assessing attention deficit hyperactivity disorder in a subject, the method comprising:
receiving EEG data and ADHD rating scale data associated with a subject;
based at least in part on a selected portion of the EEG data, determining at least one theta-beta ratio; and
based at least in part on some or all of the ADHD rating scale data, determining at least one ADHD rating scale score;
standardizing the theta-beta ratio and the at least one ADHD rating scale score; and
based at least in part on both the standardized theta-beta ratio and standardized ADHD rating scale score, determining a probability the subject has ADHD.

22. A method for assessing attention deficit hyperactivity disorder in a subject, the method comprising:
receiving EEG data from about a CZ site associated with a subject;
receiving ADHD-IV rating scale data associated with the subject;
based at least in part on a selected portion of the EEG data, determining at least one theta-beta ratio; and
based at least in part on some or all of the ADHD-IV rating scale data, determining at least one ADHD-IV rating scale score;
standardizing the theta-beta ratio and the at least one ADHD-IV rating scale score; and
determining a probability the subject has ADHD, wherein the theta-beta ratio and the at least one ADHD-IV rating scale score are entered into a logistic regression model, and an output of the model comprises a probability the subject has ADHD.

23. A system for assessing attention deficit hyperactivity disorder in a subject, the system comprising:
a data collection module operable to:
receive EEG data from about a CZ site associated with a subject;
receive ADHD-IV rating scale data associated with the subject;
a processing module operable to:
determine at least one theta-beta ratio based at least in part on a selected portion of the EEG data; and
determine at least one ADHD-IV rating scale score based at least in part on some or all of the ADHD-IV rating scale data;
standardize the theta-beta ratio and the at least one ADHD-IV rating scale score; and
determine a probability the subject has ADHD, wherein the theta-beta ratio and the at least one ADHD-IV rating scale score are entered into a logistic regression model, and an output of the model comprises a probability the subject has ADHD.
